SEQUENCE LISTING (4 sequences)

SEQ ID NO: 1 - DNA, 33996 bp, Artificial Sequence
  Description of Artificial Sequence Synthetic polynucleotide
ttcgaattaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgccggtagatcttagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcaggagctccaccgcggtggcggccgcactagtggatcccccgggctgcaggaattcaagcttatttaaatgatcgatcgatcgatcgatcgatcgatcgatcgatcgatcgatcgatcgatcgatcgatcgatcgatcgatcgatcgatcgatcgatcgatcgatcgatcgatcgatcgatcgatcgatcgatcgatttaaatatcgataccgtcgacctcgagggggggcccggatatcgtctaggtactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctcaggacagcaagggggaggattgggaagacaatagcaggcatgctggggatgcggtgggctctatgggaatcctggcgcgccggtaccggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgttgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaattcgaa

SEQ ID NO: 2 - DNA, 20 bp, Artificial Sequence
  Description of Artificial Sequence Synthetic primer
tttgggcgtaaccgagtaag

SEQ ID NO: 3 - DNA, 19 bp, Artificial Sequence
  Description of Artificial Sequence Synthetic primer
ggcgagtctccacgtaaac

SEQ ID NO: 4 - DNA, 25 bp, Artificial Sequence
  Description of Artificial Sequence Synthetic probe
agcgcgtaatatttgtctagggccg

(19) United States
(12) Patent Application Publication
(10) Pub. No.: US 2020/0208173 A1
(43) Pub. Date: Jul. 2, 2020

(54) ADENOVIRUS CONTROL VIRUS

(71) Applicant: SERACARE LIFE SCIENCES, INC., Milford, MA (US)

(72) Inventors: Bharathi Anekella, Clarksburg, MD (US); Catherine Huang, Elkridge, MD (US)

(21) Appl. No.: 15/772,301

(22) PCT Filed: Jun. 17, 2016
(86) PCT No.: PCT/US2016/037969

§ 371 Date: Apr. 30, 2018

Related U.S. Application Data
(60) Provisional application No. 62/248,812, filed on Oct. 30, 2015.

(51) Int. Cl. C12N 15/86; C12N 7/04

(52) U.S. Cl. CPC C12N 15/86; C12N 7/04; C12N 2710/10062; C12N 2710/10043; C12N 2710/10031

(57) ABSTRACT

Disclosed are compositions and methods related to replication deficient adenoviruses that are able to function as controls for nucleic acid diagnostic assays (e.g., nucleic acid sequencing based assays and/or nucleic acid amplification based assays).

### RELATED APPLICATION

This application is a U.S. National Stage Application of PCT/US2016/037969, filed Jun. 17, 2016, which claims the benefit of priority to U.S. Provisional Patent Application Ser. No. 62/248,812, filed Oct. 30, 2015.

### SEQUENCE LISTING

The instant application contains a Sequence Listing, which has been submitted electronically in ASCII format and is hereby incorporated by reference in its entirety. Said ASCII copy, created on Oct. 26, 2018, is named SCX-00501_SL.txt and is 45,364 bytes in size.

### BACKGROUND

Regulatory agencies, such as the FDA, CLIA, and CAP, generally require developers of nucleic acid-based in vitro diagnostic devices for pathogen detection to include quality controls in their regulatory submissions. Such quality control materials are important tools for the detection of analytical errors, the monitoring of long-term performance of diagnostic test kits, and the identification of changes in random or systematic error. A well-designed laboratory quality control program will generally incorporate at least some form of control that provides added confidence in the reliability of results obtained for unknown specimens.

Whole process controls are needed to monitor the entire analytical process, including sample lysis, nucleic acid extraction, amplification, detection and interpretation of results. Such controls can be natural material derived from infected patients, which have the advantage of behaving very similarly to a clinical sample. However, such natural source controls often have limited and unpredictable availability, concentration and stability. The use of cultured virus to generate positive controls alleviates some of these problems, but virus culture is often unavailable or technologically difficult. In addition, the preparation of large amount of human pathogens caries significant safety risks and is expensive.

Positive controls for amplification and detection are often provided as part of diagnostic test kits. The materials often have a known amount of input copy number and verify the integrity of the reaction components and instrument. However, such controls are not usually taken through the sample lysis or nucleic acid extraction process and are therefore unable to detect errors arising from these steps.

Internal controls contain non-target nucleic acid sequence that is co-extracted and co-amplified with the target nucleic acid. Internal controls confirm the integrity of the reagents (e.g., polymerase, primers), equipment function (e.g., thermal cycler), and the absence of inhibitors in the sample. The internal control can take the form of a non-target organism that is added to the sample prior to sample lysis and extraction. Alternatively, it could be a non-infectious, non-target DNA or RNA sequence that is added to the sample either prior to or after sample lysis and extraction.

Thus, there is a need for improved compositions able to serve as controls in diagnostic assays.

### SUMMARY

Provided herein are compositions and methods related to replication deficient adenovirus that are able to function as controls for nucleic acid diagnostic assays (e.g., nucleic acid sequencing based assays and/or nucleic acid amplification based assays).

In certain aspects, disclosed herein is a replication deficient recombinant adenovirus lacking E1 activity which comprises a genome comprising adenovirus genomic DNA sequence and a heterologous (i.e., non-adenovirus) DNA sequence. In some embodiments, the adenovirus genomic DNA sequence has a nucleic acid sequence that is at least 80%, at least 85%, at least 90%, at least 91%, at least 92%, at least 93%, at least 94%, at least 95%, at least 96%, at least 97%, at least 98%, at least 99% or 100% identical to SEQ ID NO: 1.

In some embodiments, the genome of the replication deficient adenovirus lacks a sequence encoding a functional version of one or more of an E1 protein. In some embodiments, the genome lacks a sequence encoding an E1 promoter and/or an E1 open reading frame.

In some embodiments, the heterologous DNA sequence is a DNA sequence from a non-adenovirus organism or virus. In some embodiments, the heterologous DNA sequence is a non-adenovirus DNA virus sequence. In some embodiments, the heterologous DNA sequence is a bacterial DNA sequence (e.g., a Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helobacter, Legionella, Leptospira, Listeria, Mycobacterium, Mycoplasma, Neisseria, Pseudomonas, Rickettsia, Salmonella, Shingella, Staphylococcus, Streptococcus, Treponema, Ureaplasma, Vibrio or Yersinia sequence). In some embodiments, the heterologous DNA sequence is an animal DNA sequence (e.g., a mammalian DNA sequence). In some embodiments, the heterologous DNA sequence is a human DNA sequence (e.g., the sequence of a cancer-associated gene and/or a sequence containing a cancer-associated mutation). In some embodiments, the heterologous DNA sequence is a protozoan DNA sequence (e.g., a Plasmodium, Entamoeba, Giardia, Trypanosoma, Toxoplasma, Acanthamoeba, Leishmania, Babesia, Balamuthia, Cryptosporidium, Cyclospora or Naegleria sequence). In some embodiments, the heterologous DNA sequence is a helminth DNA sequence (e.g., a Ascaris, Pinworm, Strongyloides, Toxocara, Guinea worm, hookworm, tapeworm or whipworm sequence).

In some embodiments, the heterologous DNA sequence is a bacterial DNA sequence from a bacterium selected from the group consisting of Bacillus anthraces, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

In some embodiments, the heterologous DNA sequence is a pathogen DNA sequence from a pathogen selected from the group consisting of Trichomonas vaginalis, Candida Albicans, Plasmodium falciparum, Leishmania, Trypanosomes, Ascariasis, Babesia, Shistosomes , Cyclospora cayetanensis, Cryptosporidium parvum and Toxoplasma.

In some embodiments, the heterologous DNA sequence includes at least 10, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 bp of a non-adenovirus organism or virus sequence. In some embodiments, the heterologous DNA sequence includes 100-300 bp of a non-adenovirus organism or virus sequence. In some embodiments, the heterologous DNA sequence includes 100-200 bp of a non-adenovirus organism or virus sequence. In some embodiments, the heterologous DNA sequence is at least 80%, at least 85%, at least 90%, at least 91%, at least 92%, at least 93%, at least 94%, at least 95%, at least 96%, at least 97%, at least 98%, at least 99% or 100% identical to a non-adenovirus organism or virus sequence. In some embodiments, the non-adenovirus organism or virus sequence comprises one or more mutations. In some embodiments, the mutation is an insertion, a deletion and/or a substitution (e.g., a single nucleotide polymorphism).

In some embodiments, the heterologous DNA sequence in the genome comprises a non-adenovirus DNA virus sequence. In some embodiments, the non-adenovirus DNA virus sequence is a dsDNA virus sequence. In some embodiments, the non-adenovirus DNA virus sequence is a ssDNA virus sequence. In some embodiments, the heterologous DNA sequence includes at least 10, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 bp of a non-adenovirus DNA virus sequence. In some embodiments, the heterologous DNA sequence includes 100-300 bp of a non-adenovirus DNA virus sequence. In some embodiments, the heterologous DNA sequence includes 100-200 bp of a non-adenovirus DNA virus sequence. In some embodiments, the heterologous DNA sequence is at least 80%, at least 85%, at least 90%, at least 91%, at least 92%, at least 93%, at least 94%, at least 95%, at least 96%, at least 97%, at least 98%, at least 99% or 100% identical to a non-adenovirus DNA virus sequence. In some embodiments, the non-adenovirus DNA virus sequence comprises one or more mutations. In some embodiments, the mutation is an insertion, a deletion and/or a substitution (e.g., a single nucleotide polymorphism). In some embodiments, the mutation conveys a drug resistant phenotype when present in the non-adenovirus DNA virus. For example, in some embodiments the non-adenovirus DNA virus sequence comprises at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 mutations that convey a drug resistant phenotype when present in the non-adenovirus DNA virus.

In some embodiments, the heterologous DNA sequence comprises a non-adenovirus DNA virus sequence. In some embodiments, the non-adenovirus DNA virus sequence is a variola sequence (e.g., a variola major sequence and/or a variola minor sequence), a herpesviridae sequence (e.g., an HHV-1 sequence, an HHV-2 sequence, an HHV-3 sequence, an HHV-4 sequence, an HHV-5 (i.e., CMV) sequence, an HHV-6 sequence, an HHV-8 sequence), a papillomavirus sequence (e.g., an HPV sequence), a polyomavirus sequence (e.g., a BK virus sequence, a JC virus sequence), a hepatitis B virus sequence and/or a parovirus sequence.

In some embodiments, the heterologous DNA sequenc comprises a non-adenovirus DNA sequence from a human pathogen. Such pathogens may include eukaryotic protozoan such as Trypanosomes, Leishmania and Toxoplasma parasites. Such pathogens may also include multicellular parasites such as shistosomes. Additionally in some embodiments, the heterologous DNA sequence could be from a prokaryote such as Listeria, or Yersinia or other pathogenic bacteria.

In some embodiments, the heterologous DNA sequence is an internal control sequence. An internal control sequence can be any non-target nucleic acid sequence (artificial or natural) that is co-extracted and co-amplified with the target nucleic acid during a nucleic acid-based assay. Internal controls confirm the integrity of the reagents (e.g., polymerase, primers), equipment function (e.g., thermal cycler), and the absence of inhibitors in the sample. In some embodiments, the internal control sequence is the sequence from a non-target organism not normally present in the sample being tested. In some embodiments, the internal control sequence is an artificial sequence not found in nature. In some embodiments, the internal control sequence is at least 10, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 bp in length.

In certain aspects, provided herein is a composition comprising a replication deficient adenovirus described herein. In certain aspects, the composition comprises two or more of the replication deficient adenoviruses described herein. In some embodiments, the composition further comprises human DNA. In some embodiments, the replication deficient adenovirus is in a human bodily fluid. In some embodiments, the human bodily fluid is human plasma (e.g., difibrinated human plasma). In some embodiments, the composition further comprises a preservative, such as sodium azide. In some embodiments, the replication deficient adenovirus is associated with a cell, tissue or cell line. In some embodiments, the cell, tissue or cell line is fixed.

In certain aspects, provided herein is a nucleic acid molecule encoding the DNA genome of the replication deficient adenovirus described herein. In some embodiments, the nucleic acid molecule is a DNA molecule. In some embodiments, the nucleic acid molecule is a plasmid (e.g., a circular plasmid or a linearized plasmid, such as a circular expression plasmid or a linearized expression plasmid). In some embodiments, the nucleic acid molecule is isolated. In certain embodiments, provided herein is a cell comprising a nucleic acid described herein. In some embodiments, the cell is a HEK 293 cell.

In certain aspects, provided herein is a method of making a replication deficient adenovirus. In certain embodiments, the method includes the step of transfecting a cell that expresses adenovirus E1 protein (e.g., a HEK 293 cell that expresses adenovirus E1 protein) with a nucleic acid molecule (e.g., a linearized plasmid molecule) encoding the genome of a replication deficient adenovirus described herein. In some embodiments, the cell is then cultured under conditions such that the cell produces the replication deficient adenovirus. In some embodiments, the method further comprises collecting the replication deficient adenovirus (e.g., by collecting the culture supernatant). In some embodiments, the method comprises amplifying the replication deficient adenovirus by infecting a cell that expresses adenovirus E1 protein (e.g., a HEK 293 cell that expresses adenovirus E1 protein) with a replication deficient adenovirus described herein. In some embodiments, the infected cell is then cultured under conditions such that the cell produces the replication deficient adenovirus. In some embodiments, the method further comprises collecting the replication deficient adenovirus (e.g., by collecting the culture supernatant). In some embodiments, the method further comprises filtering and/or heat inactivating the culture supernatant. In some embodiments, the method further comprises determining the titer of the virus (e.g., using real-time PCR).

In certain aspects, provided herein are methods of testing a diagnostic assay by running the diagnostic assay on a composition comprising the replication deficient adenovirus described herein. In some embodiments, the diagnostic assay is a nucleic acid amplification based diagnostic assay. In some embodiments, the diagnostic assay is a sequencing based diagnostic assay. In some embodiments the diagnostic assay is an assay for the detection of a DNA virus. In some embodiments, the diagnostic assay is an assay for the detection of a variola virus (e.g., a variola major virus and/or a variola minor virus), a herpesviridae virus (e.g., an HHV-1 virus, an HHV-2 virus, an HHV-3 virus, an HHV-4 virus, an HHV-5 virus, an HHV-6 virus, an HHV-8 virus), a papillomavirus virus (e.g., an HPV virus), a polyomavirus virus (e.g., a BK virus, a JC virus), a hepatitis B virus and/or a parovirus virus. In certain embodiments, the heterologous DNA sequence in the genome of the replication deficient adenovirus contains the target sequence detected in the diagnostic assay. In some embodiments, the method includes the performance of a sample lysis step on the composition comprising the replication deficient adenovirus. In some embodiments, the method comprises performing a nucleic acid extraction step. In some embodiments, the method comprises performing a nucleic acid amplification step (e.g., performing a real-time nucleic acid amplification/detection process). In some embodiments, the method comprises performing a nucleic acid sequencing step. In some embodiments the method comprises performing a nucleic acid detection step.

### BRIEF DESCRIPTION OF FIGURES

FIG. 1 shows a schematic depiction of the organization of an exemplary adenovirus virus vector. SOI is the Sequence Of Interest.

FIG. 2 shows an exemplary schematic for the production of recombinant Adenovirus control viruses.

FIG. 3 shows the detection of an internal control adenovirus vector in a viral load assay. A recombinant adenovirus was produced that bears an internal control sequence (a sequence that is amplified by the pathogen detection primers, but has a unique sequence recognized by an independent probe). Ten replicates of 1:100 diluted stock were tested via TaqMan® real time assay across multiple runs to demonstrate consistency of the internal control.

FIG. 4 provides the nucleic acid sequence of an exemplary adenovirus victor (SEQ ID NO: 1).

### DETAILED DESCRIPTION

### General

Provided herein are compositions and methods related to replication deficient adenovirus that are able to function as controls for nucleic acid diagnostic assays (e.g., nucleic acid sequencing based assays and/or nucleic acid amplification based assays). In certain aspects, provided herein are adenovirus control virus useful as whole process controls, positive controls and/or internal controls in nucleic acid diagnostic assays. Such control virus can benefit diagnostics manufacturers by providing a less expensive, consistent and safe source of starting material for controls. Moreover, the adenovirus structure also confers stability to the encapsulated DNA in complex biological matrices such as plasma, blood or urine. The control virus described herein use adenovirus virus, an DNA containing virus that can be engineered to contain target DNA sequences, such as sequences from another virus and/or an internal control sequence. In some embodiments, the recombinant adenovirus system described herein results in viral particles that are packaged, so they can be used to evaluate nucleic acid extraction processes that are used before nucleic acid detection. Also provided herein are compositions comprising such viruses, nucleic acid molecules encoding the genome of such control viruses, methods of making such control viruses and methods of using such control viruses.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “control” includes any portion of an experimental system designed to demonstrate that the factor being tested is responsible for the observed effect, and is therefore useful to isolate and quantify the effect of one variable on a system.

The term “gene” is used broadly to refer to any nucleic acid associated with a biological function. The term “gene” applies to a specific genomic sequence, as well as to a cDNA or an mRNA encoded by that genomic sequence.

As used herein, the term “heterologous DNA” refers to DNA present in a recombinant adenovirus that is not derived from wild-type adenovirus. For example, heterologous DNA in a adenovirus virus can be a DNA sequence normally found in a different virus (e.g., a different DNA virus), can be an DNA sequence normally found a non-viral organism, or can be a completely artificial DNA sequence.

The term “isolated nucleic acid” refers to a polynucleotide of natural or synthetic origin or some combination thereof, which (1) is not associated with the cell in which the “isolated nucleic acid” is found in nature, and/or (2) is operably linked to a polynucleotide to which it is not linked in nature.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, the term “adenovirus” includes medium-sized (90-100 nm) nonenveloped viral particles made up of an icosahedral nucleocapsid that comprises adenovirus virus capsid proteins encompassing a double-stranded DNA genome. The DNA genome can include non-adenovirus DNA (i.e., heterologous DNA) and does not need to include all parts of the wild-type adenovirus genome. For example, in some embodiments the DNA genome does not encode functional adenovirus E1 protein.

### Replication Deficient Adenovirus Control Viruses

In certain embodiments, provided herein are replication deficient Adenovirus control viruses. In some embodiments, such viruses have an dsDNA genome that includes (a) sequences encoding adenovirus proteins and (b) a heterologous (i.e., non-adenovirus) DNA sequence. In some embodiments, the heterologous DNA sequence is a sequence from a different DNA virus, such as a variola virus (e.g., a variola major virus and/or a variola minor virus), a pox virus (e.g., small pox, cowpox, vaccinia virus), a herpesviridae virus (e.g., an HHV-1 virus, an HHV-2 virus, an HHV-3 virus, an HHV-4 virus, an HHV-5 virus, an HHV-6 virus, an HHV-8 virus), a papillomavirus virus (e.g., an HPV virus), a polyomavirus virus (e.g., a BK virus, a JC virus), a hepatitis B virus and/or a parovirus virus (e.g., parovirus B19). In certain embodiments, the heterologous DNA sequence is a sequence of a herpes virus modified for viral-based cancer therapy (e.g., a sequence from a genetically engineered virus called talimogene laherparepvec (T-VEC) to treat advanced melanoma).

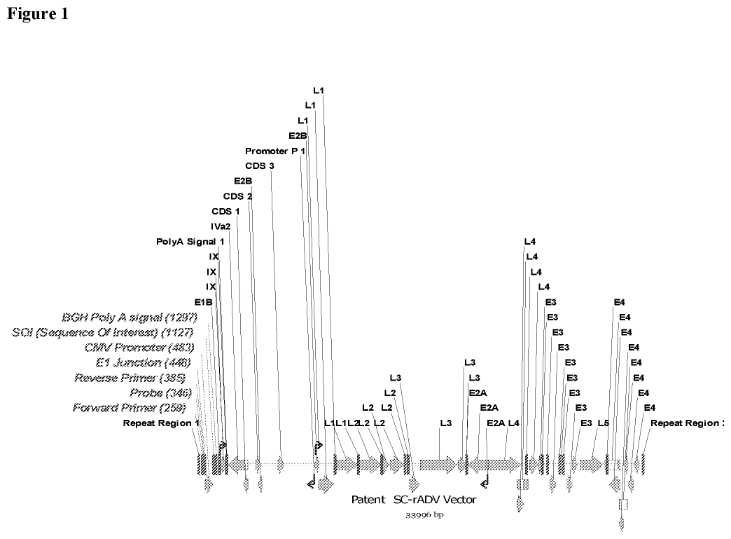

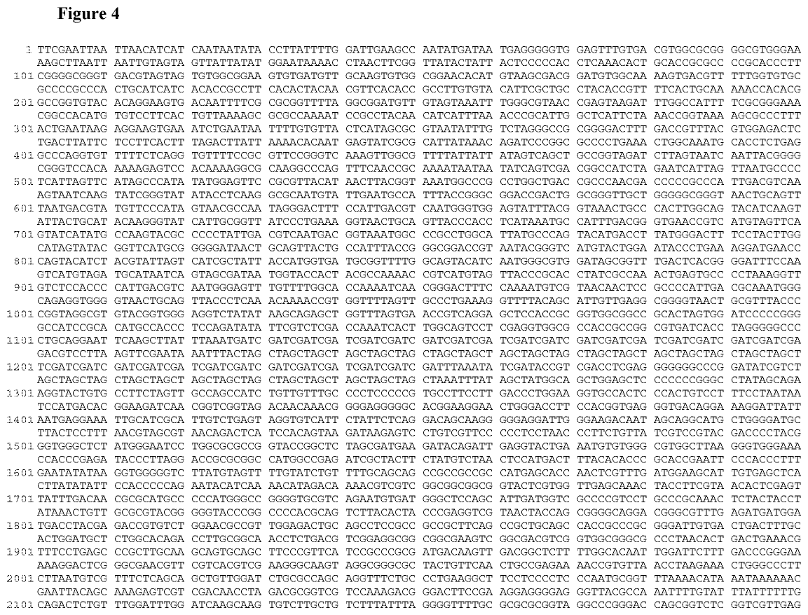

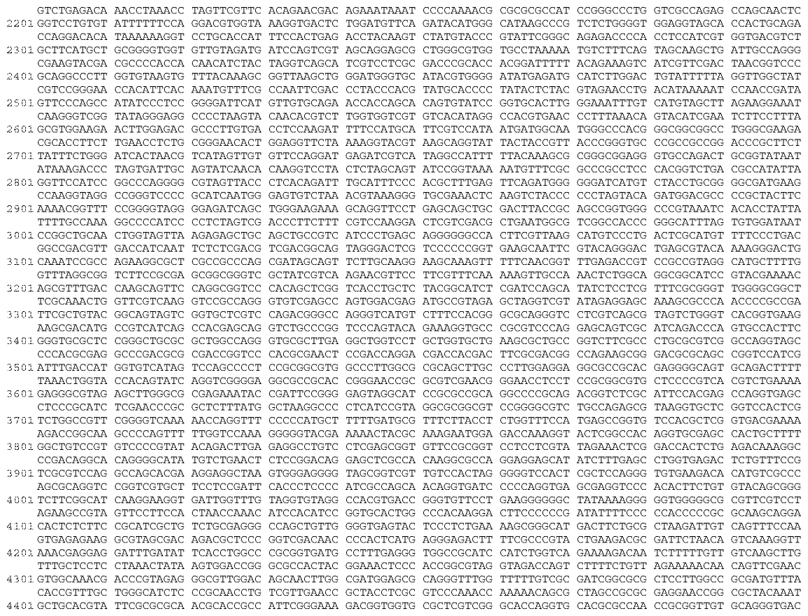

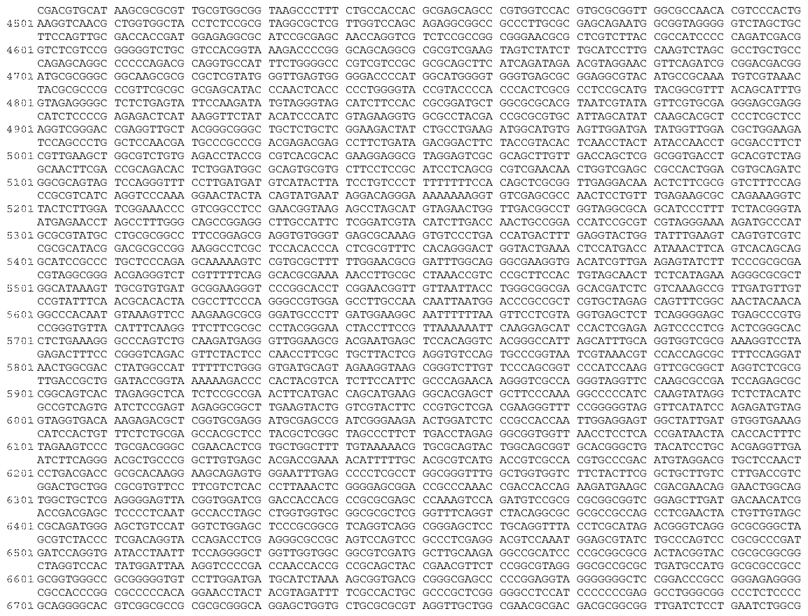

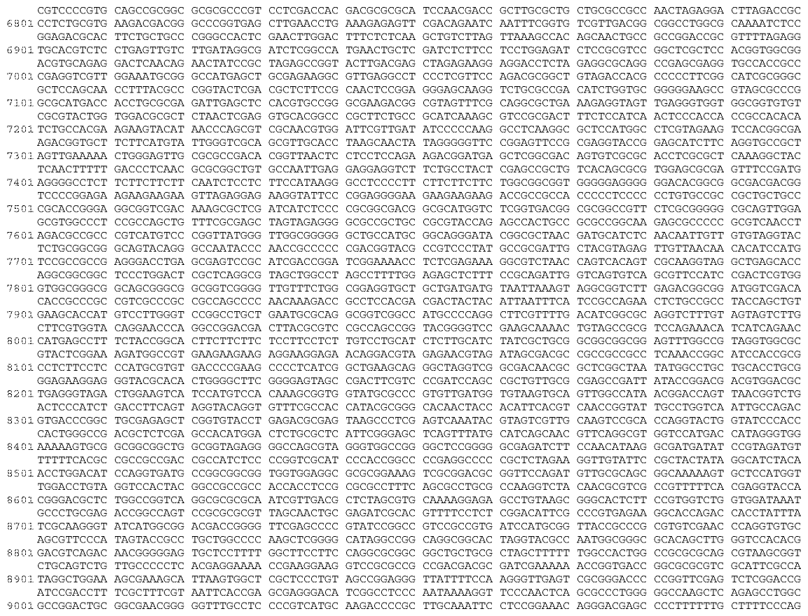

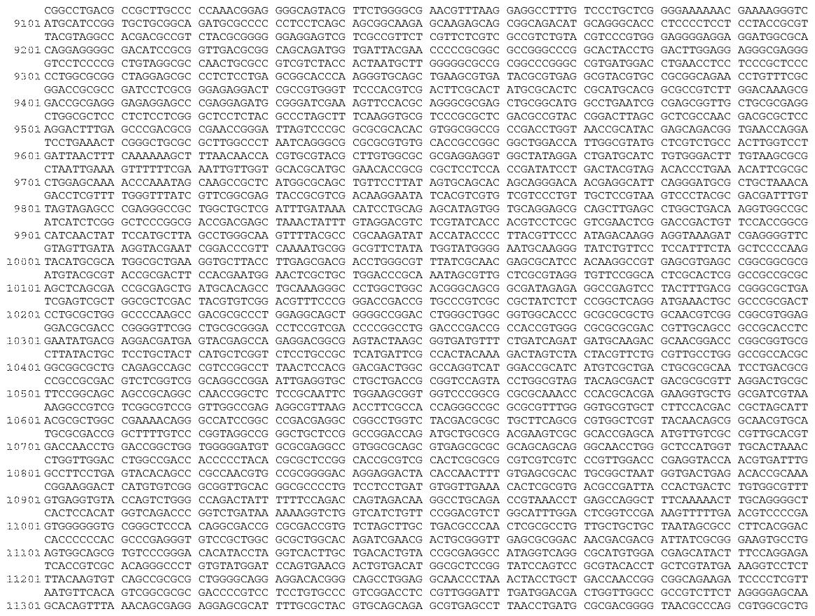

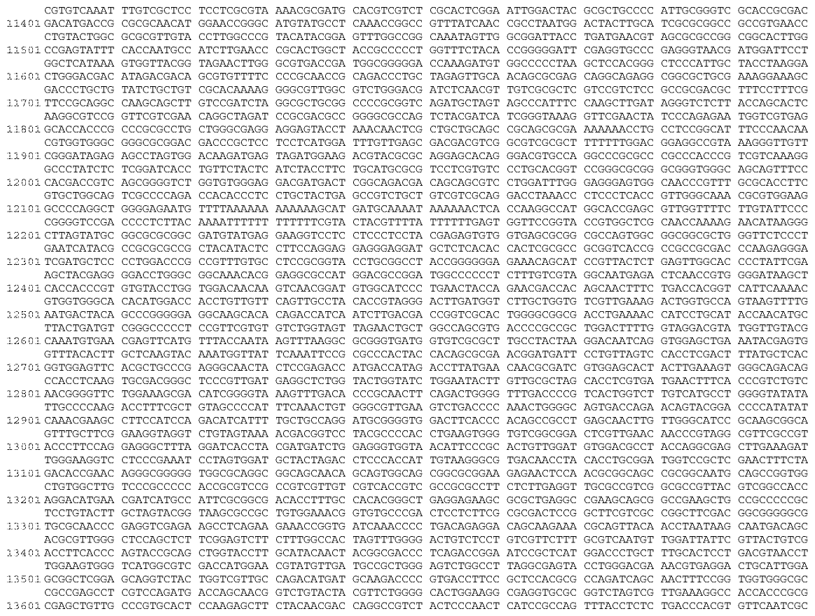

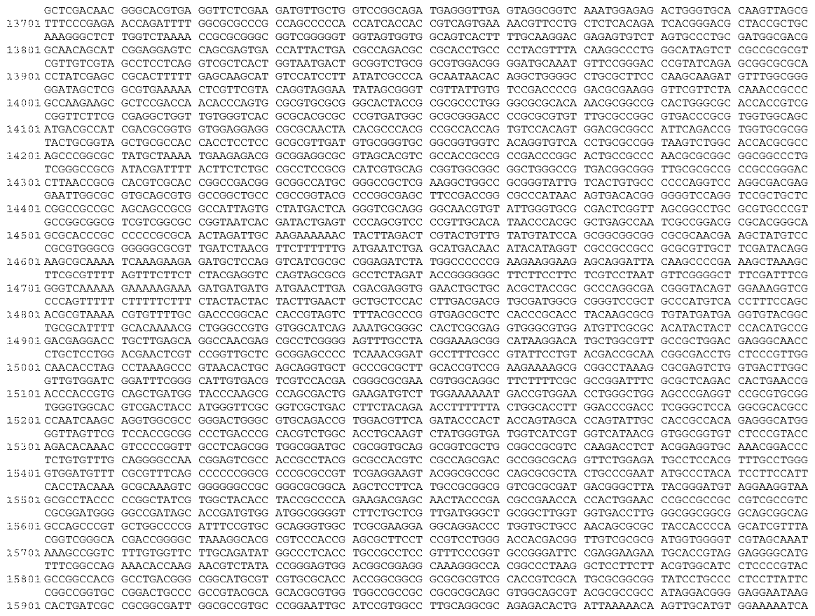

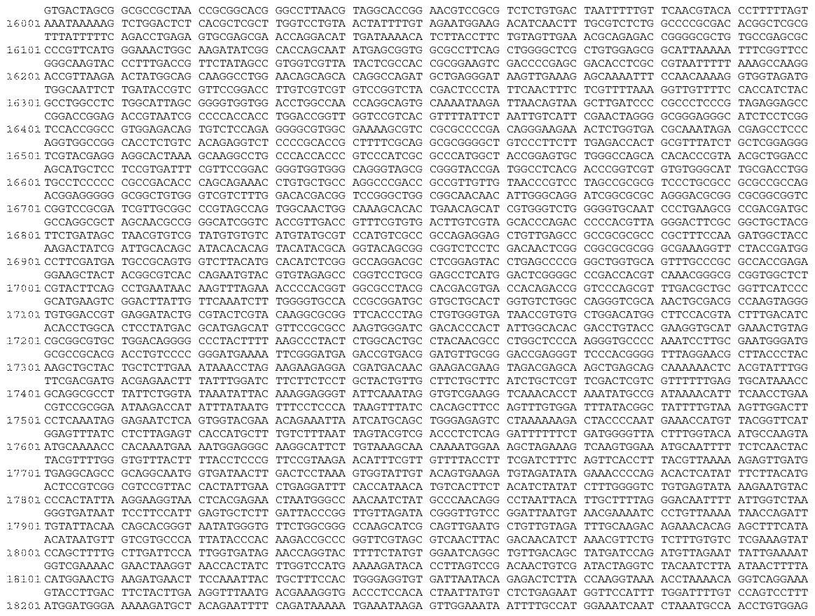

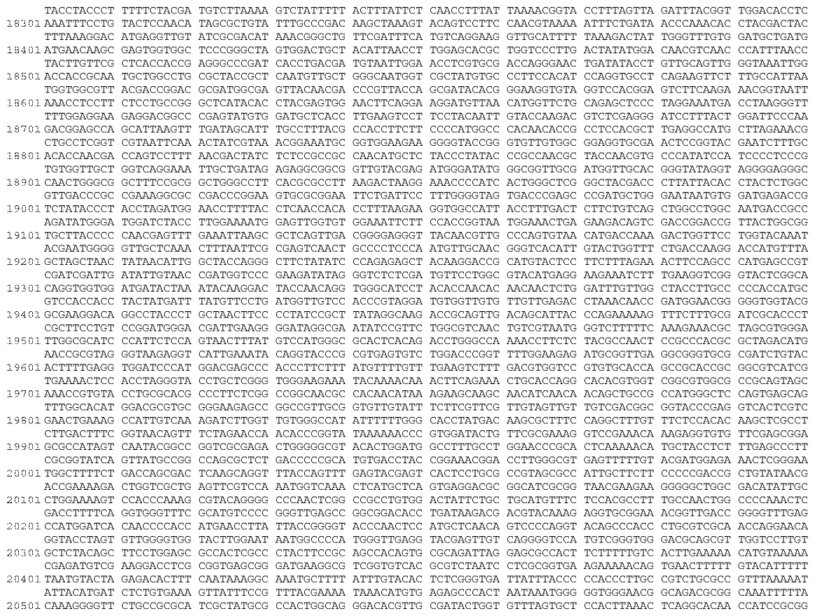

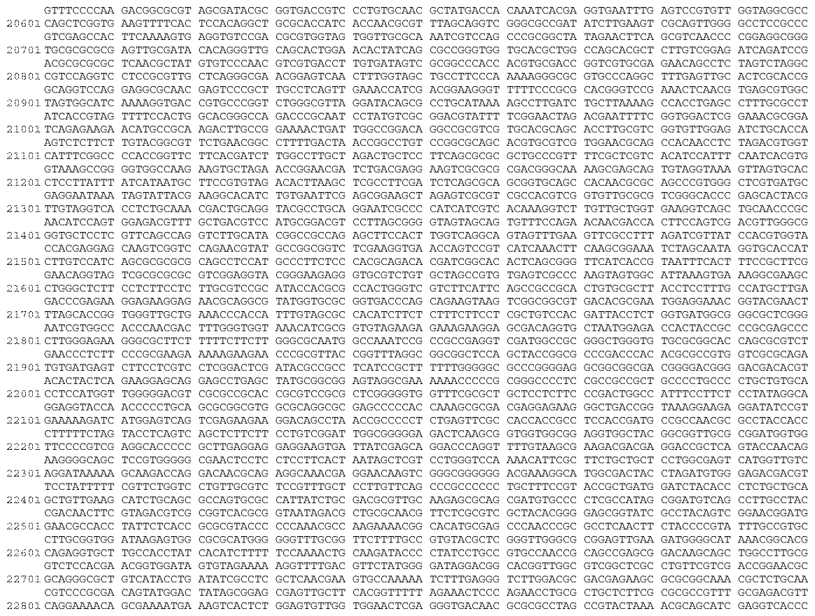

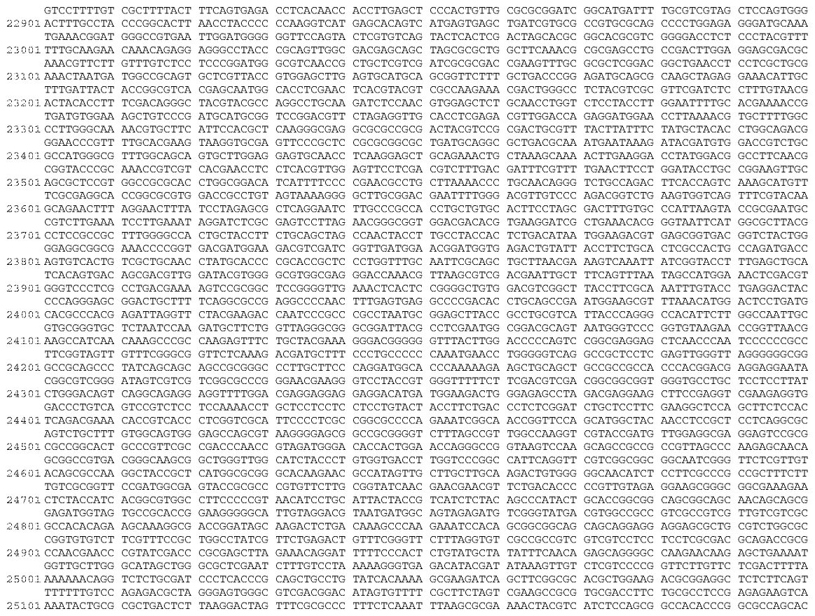

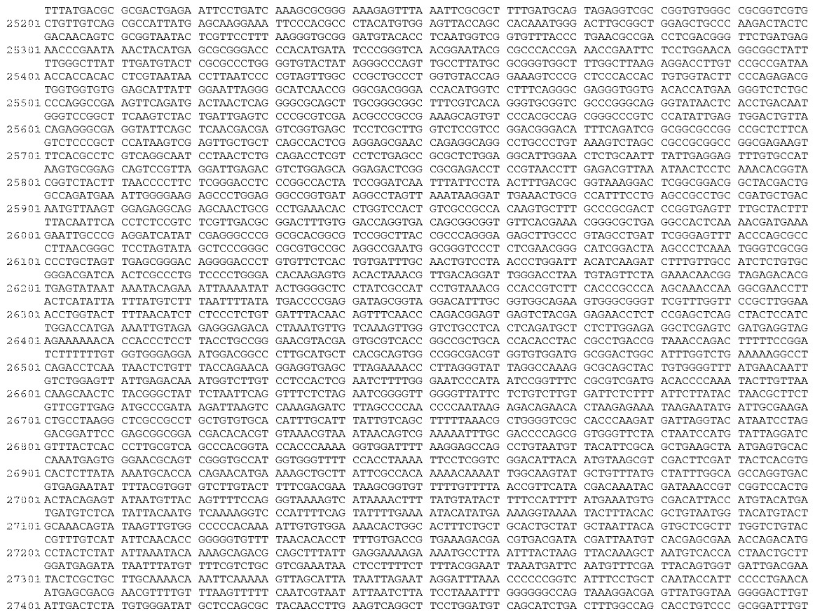

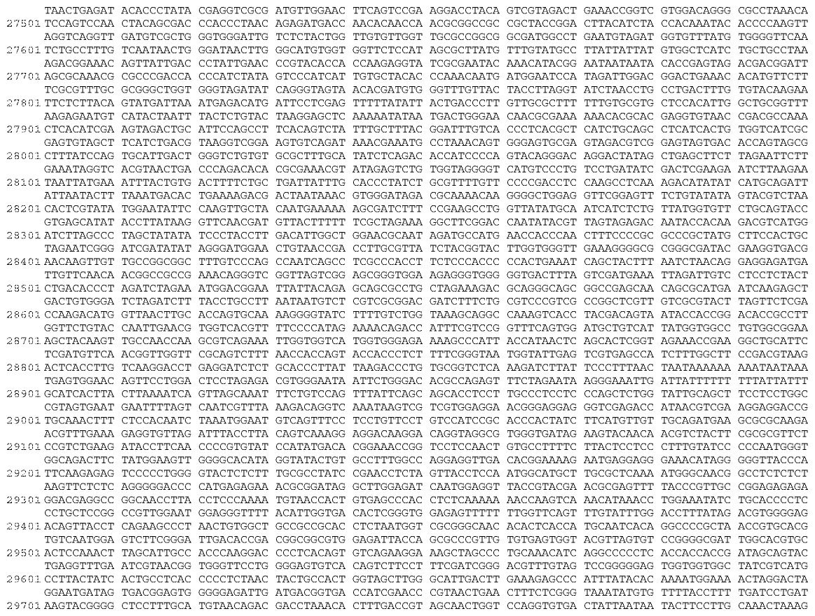

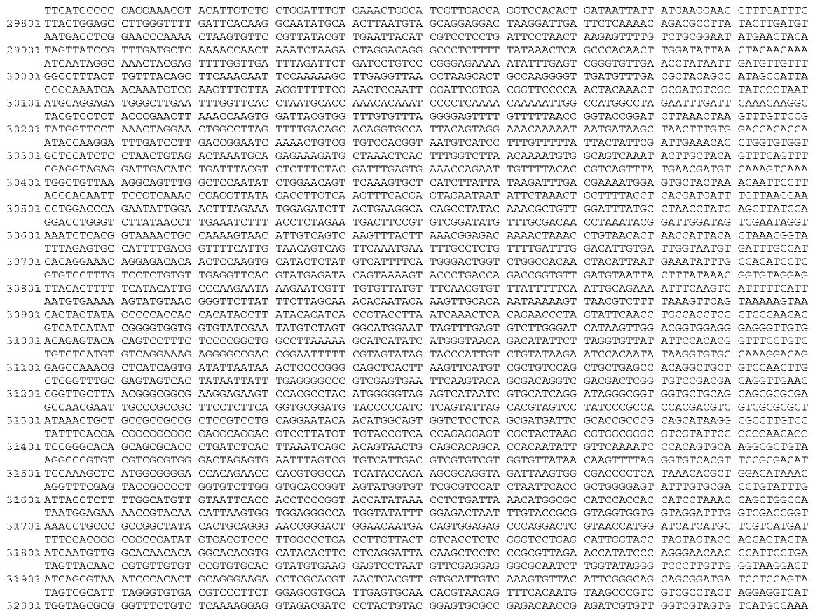

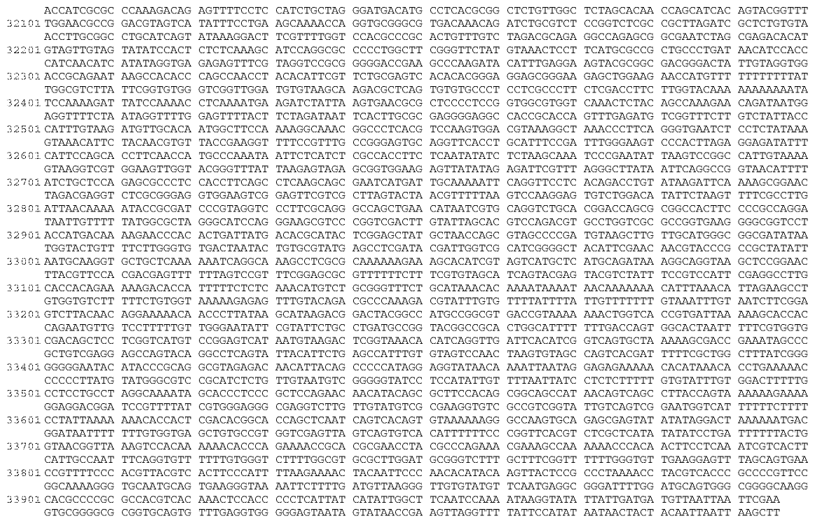

In certain embodiments, the recombinant adenovirus control viruses described herein are replication deficient. In some embodiments, any method can be used to render the recombinant adenovirus control virus replication deficient. In some embodiments the adenovirus control virus does not encode one or more functional structural proteins. In some embodiments, the recombinant adenovirus control virus genome does not encode one or more functional nonstructural proteins. In some embodiments, the adenovirus control virus does not encode a functional E1 protein. In some embodiments, the adenovirus control virus does not encode a functional E1 promoter. FIG. 1 depicts a map of an exemplary adenovirus control virus lacking an E1 promoter and protein coding sequence. An exemplary sequence of such an adenovirus is provided in FIG. 4.

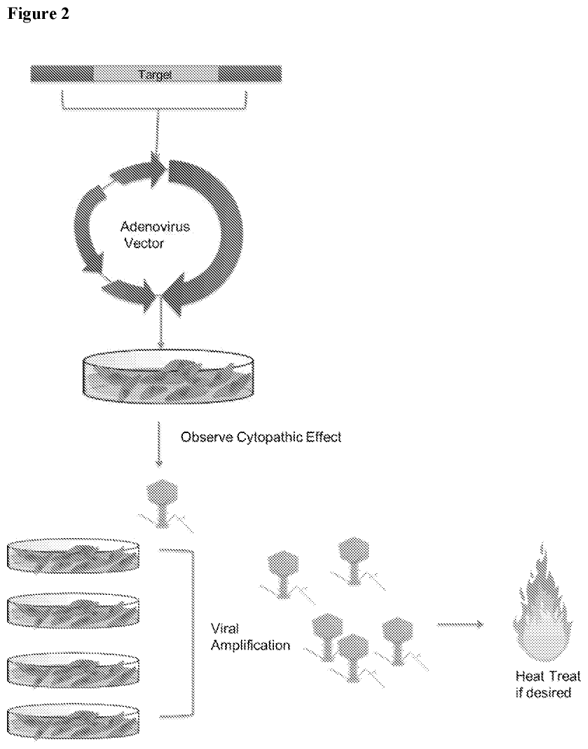

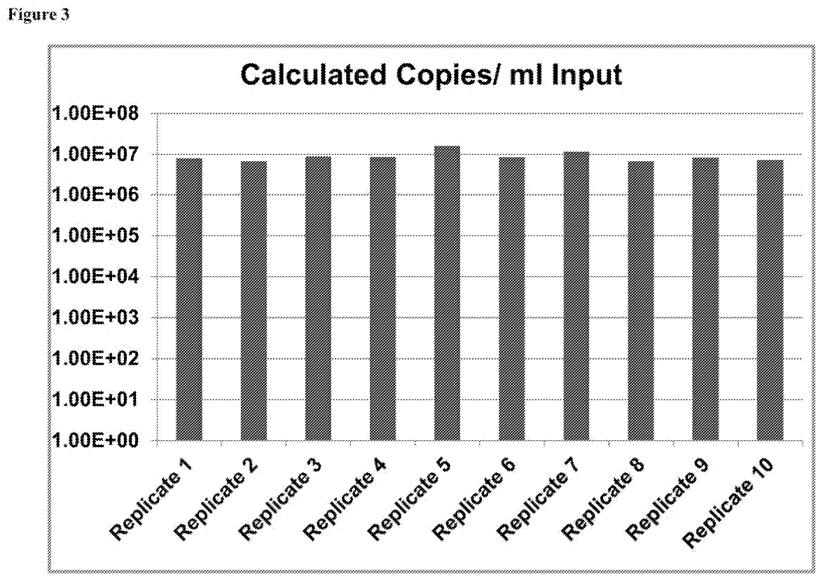

The Adenovirus control viruses described herein can be generated using any method known in the art. An exemplary method of generating the Adenovirus control viruses described herein is illustrated in FIG. 2. In this exemplary method, the target sequence is cloned into an adenovirus vector that lacks the adenovirus E1 promoter and ORF. The linearized plasmid is introduced into E1 complementing cells: only cells expressing E1 in trans support viral replication. When cytopathic effect is observed, cell lysate is generated. The virus can then be further amplified, purified, and heat treated (if desired). The viral particles produced are replication defective because they lack the critical E1 gene.

### Use of Adenovirus Control Vectors in Nucleic Acid Diagnostic Assays

In certain aspects, provided herein are methods of testing a diagnostic assay by running the diagnostic assay on a composition comprising the replication deficient adenovirus described herein. In some embodiments, the diagnostic assay is an assay for the detection of a variola virus (e.g., a variola major virus and/or a variola minor virus), a herpesviridae virus (e.g., an HHV-1 virus, an HHV-2 virus, an HHV-3 virus, an HHV-4 virus, an HHV-5 virus, an HHV-6 virus, an HHV-8 virus), a papillomavirus virus (e.g., an HPV virus), a polyomavirus virus (e.g., a BK virus, a JC virus), a hepatitis B virus and/or a parovirus virus. In certain embodiments, the heterologous DNA sequence in the genome of the replication deficient adenovirus virus contains the target sequence detected in the diagnostic assay.

In some embodiments, the diagnostic assay is a nucleic acid amplification based diagnostic assay. In some embodiments, the nucleic acid amplification based diagnostic assay includes a sample lysis step, a nucleic acid extraction step (e.g., a magnetic-bead based nucleic acid extraction step), a nucleic acid amplification step and/or a nucleic acid detection step. In some embodiments, the nucleic acid amplification and detection steps are performed simultaneously (e.g., through the use of a real-time detection technology, such as TaqMan probes or molecular beacons). Examples of nucleic acid amplification processes include, but are not limited to, polymerase chain reaction (PCR), ligase chain reaction (LCR), strand displacement amplification (SDA), transcription mediated amplification (TMA), self-sustained sequence replication (3SR), Qβ replicase based amplification, nucleic acid sequence-based amplification (NASBA), repair chain reaction (RCR), boomerang DNA amplification (BDA) and/or rolling circle amplification (RCA).

In some embodiments, the diagnostic assay is a nucleic acid sequencing based diagnostic assay (e.g., a next-generation sequencing based diagnostic assay). In some embodiments, the nucleic acid sequencing based diagnostic assay includes a sample lysis step, a nucleic acid extraction step (e.g., a magnetic-bead based nucleic acid extraction step), a nucleic acid amplification step, and/or a nucleic acid sequencing step. Examples of nucleic acid sequencing processes include, but are not limited to chain termination sequencing, sequencing by ligation, sequencing by synthesis, pyrosequencing, ion semiconductor sequencing, single-molecule real-time sequencing, 454 sequencing, and/or Dilute-‘N’-Go sequencing.

### EXAMPLES

### Example 1

### Production of an Adenovirus Control Virus

Wild type Adenovirus Type 5 AY601635 (Ad5) sequence was used as the as base for construction of an adenovirus control virus. The Ad5 sequence was modified and the recombinant clone synthesized using DNA 2.0 gene construction service. So that the adenovirus grows only on complementing cell lines, E1 deleted adenovirus was generated that can be grown only in E1 complementing cell line but not in any other human or nonhuman cell lines. The following changes were made to the Ad5 sequence (vector map provided in FIG. 1, sequence provided in FIG. 4):
a. E1 promoter and open reading frames were deleted including the base pairs 458-3482
b. Pac I and BstB1 sites were introduces at the beginning and end of the clone. So digestion with either one of the enzyme will release the entire clone from the plasmid and can be used for transfection.
c. CMV promoter sequence is introduced followed by unique RE site. This feature can be used for expression if required in future.
d. Introduced a Swal unique site so sequence of interest (SOI) can be cloned as needed.
e. Following SwaI site multiple cloning sites were introduced followed by a BGH Poly A signal sequence.
f. Deleted additional XbaI site in the VA region (10589 site) so this enzyme site can be used for future manipulations.

An adenovirus vector constructed as described above and bearing a cloned sequence of interest was grown and amplified in bacteria and the plasmid DNA was purified using the Qiagen Plasmid Maxi/Mega kit. The isolated DNA was then digested with PacI restriction enzyme, which exposes the viral inverted terminal repeats (ITRs). After PacI digestion, the DNA was purified by extraction with phenol:chloroform and then precipitated with isopropyl alcohol. The purified DNA was resuspended in molecular biology grade water.

Human Embryonic Kidney (HEK) 293 cells were obtained from ATCC (catalog #CRL-1573™). One working stock vial was removed from cryostorage and grown in Eagle's Minimum Essential Medium (EMEM) with 10% fetal bovine serum. The cells were passaged two to four times prior to transfection. Cell growth and viability was monitored by hemacytometer and the viability at each passage was greater than 90%. The day before transfection, the cells were plated at 5×10⁵ cells per well in a 6-well plate in 2 mL of EMEM Complete Growth Media. On the day of transfection, DNA-Lipofectamine™ 2000 (Invitrogen) complexes for each transfection sample were prepared according to the Lipofectamine 2000 manufacturer's instructions (1 μg of linear DNA fragment was used per transfection). The cells were monitored by light microscopy for visible regions of cytopathic effect (CPE) which were typically observed 7-10 days post-transfection. When Cytopathic effect reached 80%, the cells were harvested and a crude lysate was made by repetitive cycles of freezing and thawing the cells.

To amplify the virus, HEK 293 cells were grown in culture, using a new working stock vial if necessary to approximately 80% confluency. The day before infections, cells were seeded in a 10 cm tissue culture plate at approximately 3×10⁶ cells per plate. On the day of infection, approximately 100-200 μL of crude adenoviral lysate was added to the cells, which corresponds to a multiplicity of infection (MOI) of 3-5. The cells were incubated the cells at 37° C. in a CO₂ incubator and the infection was allowed to proceed until 80-90% of the cells have rounded up and were floating (typically 2-3 days post-infection). This indicated that cells are loaded with adenoviral particles. The cells were harvested and a crude lysate was made by repetitive cycles of freezing and thawing the cells.

To purify the viral particles, the amplified viral lysate was centrifuged for 5 minutes at top speed in a benchtop microcentrifuge and the clarified supernatant was collected. 1 μL of 25 U/μL) of benzonase enzyme was added to every 1 mL of viral supernatant. The benzonase reaction was incubated at 37° C. for 30 minutes. Purification used ViraPur Adenovirus Standard Purification Virakit following the manufacturer's instruction. 10 mL of crude lysate was mixed with an equal volume of the kit dilution buffer and passed over a single column. Elution from the column was performed using the kit elution buffer, and then the eluate was mixed with glycerol (to 10% final concentration) before aliquotting and freezing.

The final product was titered using a TaqMan Real time PCR Assay. The primers and probes for this assay are given in table 1. The PCR standards are made from a plasmid which contains adenovirus sequences and the primers and probes targets this sequence. The linearized plasmid was quantitated by PicoGreen Assay (Molecular Probes/Life Technologies), and diluted appropriately.

TABLE 1
Primers and probe for TaqMan based adenovirus
quantitation assay.

| | | SEQ | |
|---|---|---|---|
| | | ID | |
| Name | Sequence | NO: | Function |
| Adeno | 5′- | 2 | Adenovirus |
| Vector- | TTTGGGCGTAACCGAGTAAG-3′ | | Vector |
| F | | | sequence |
| | | | Forward |
| | | | primer |
| Adeno | 5′- | 3 | Adenovirus |
| Vector- | GGCGAGTCTCCACGTAAAC-3′ | | Vector |
| R | | | sequence |
| | | | Reverse |
| | | | primer |
| Adeno | 5′-6FAM- | 4 | Adenovirus |
| Vector | AGCGCGTAATATTTGTCTAGGG | | Vector |
| probe | CCG-BHQ1-3′ | | sequence |
| | | | probe |

Purified Adenovirus lot 13110 was tested at zero months, 21 and 25 Month time points to assign 2 year dating when stored at −70° C. Except for the zero time point, the remaining two time points was tested in 3 replicates on in-house developed TaqMan assay as per SOP19162 (see Table 1). Zero Time point was tested in 5 replicates on two different runs for release testing. At each time point virus was diluted at 1:100 in PBS before testing. Data for all the time points is presented below.

TABLE 2
Real Time stability data indicating 2 year shelf life at −70° C.

| Time (months) | Viral Count (copies/ml) |
|---|---|
| 0 | 1.45 × 10⁹ |
| 0 | 3.12 × 10⁸ |
| 0 | 8.80 × 10⁸ |
| 20 | 5.09 × 10⁸ |
| 25 | 3.01 × 10⁹ |

Data suggests that no reduction in copies/mL of virus is observed after storing virus for 2 years at −70 V temperature. With this data two year dating can be assigned to the Purified Adenovirus lots.

To test the use of the adenovirus control vector as a control, the recombinant adenovirus that bears an internal control sequence was produced as described above (a sequence that is amplified by the pathogen detection primers, but has a unique sequence recognized by an independent probe). Ten replicates of 1:100 diluted stock were tested via TaqMan® real time assay across multiple runs to demonstrate consistency of the internal control. The results of the detection assay are depicted in FIG. 2.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A replication deficient recombinant adenovirus lacking E1 activity, comprising a genome comprising adenovirus genomic DNA sequence and a heterologous DNA sequence.

2. The replication deficient recombinant adenovirus of claim 1, wherein the adenovirus genomic DNA sequence is at least 90% identical to SEQ ID NO: 1.

3. The replication deficient recombinant adenovirus of claim 1, wherein the adenovirus genomic DNA sequence comprises a nucleic acid sequence of SEQ ID NO: 1.

4. The replication deficient recombinant adenovirus of claim 1, wherein the genome lacks a sequence encoding an E1 promotor or an E1 open reading frame.

5. The replication deficient recombinant adenovirus of claim 1, wherein the heterologous DNA sequence comprises a non-adenovirus organism or virus sequence.

6. The replication deficient recombinant adenovirus of claim 5, wherein the heterologous DNA sequecne comprises DNA from the genome of a human pathogen.

7. The replication deficient recombinant adenovirus of claim 6, wherein the human pathogen is a protozoan, a multicellular parasite or a bacterium.

8. The replication deficient recombinant adenovirus of claim 5, wherein the heterologous DNA sequence is a human DNA sequence.

9. The replication deficient recombinant adenovirus of claim 8, wherein the human DNA sequence comprises the sequence of a cancer-associated gene or mutation.

10. The replication deficient recombinant adenovirus of claim 5, wherein the heterologous DNA sequence is a non-adenovirus DNA virus sequence.

11. The replication deficient recombinant adenovirus virus of claim 10, wherein the non-adenovirus DNA virus sequence comprises one or more mutations that convey drug resistance when they occur in the non-adenovirus DNA virus.

12. The replication deficient recombinant adenovirus virus of claim 10, wherein the non-adenovirus DNA virus sequence comprises at least 5 mutations that convey drug resistance when they occur in the non-adenovirus DNA virus.

13-15. (canceled)

16. The replication deficient recombinant adenovirus virus of claim 1, wherein the heterologous DNA sequence is an internal control sequence.

17. (canceled)

18. (canceled)

19. A composition, comprising a replication deficient adenovirus of claim 1.

20-26. (canceled)

27. A nucleic acid molecule encoding the genome of the replication deficient adenovirus of claim 1.

28. The nucleic acid molecule of claim 27, wherein the nucleic acid molecule is a DNA molecule.

29. The nucleic acid molecule of claim 28, wherein the DNA molecule is a plasmid.

30. The plasmid of claim 29, wherein the plasmid is a linearized plasmid.

31. A method of making a replication deficient Adenovirus virus comprising:
(a) transfecting a cell that expresses adenovirus E1 proteins with the linearized plasmid molecule of claim 30;
(b) culturing the transfected cell of step (a) under conditions such that the cell produces a replication deficient adenovirus; and
(c) collecting the replication deficient adenovirus.

32-34. (canceled)

35. A method of testing a diagnostic assay, comprising performing the diagnostic assay on a composition of claim 19.
